(19) United States
(12) Patent Application Publication
(10) Pub. No.: US 2023/0000928 A1
(43) Pub. Date: Jan. 5, 2023

(54) PAR2 MODULATION TO ALTER MYELINATION

(71) Applicant: Mayo Foundation for Medical Education and Research, Rochester, MN (US)

(72) Inventors: Hye-Sook Yoon, Rochester, MN (US); Isobel A. Scarisbrick, Rochester, MN (US)

(73) Assignee: Mayo Foundation for Medical Education and Research, Rochester, MN (US)

(21) Appl. No.: 17/944,699

(22) Filed: Sep. 14, 2022

Related U.S. Application Data
(62) Division of application No. 16/312,411, filed on Dec. 21, 2018.
(60) Provisional application No. 62/353,769, filed on Jun. 23, 2016.

(51) Int. Cl. A61K 35/545; C12N 5/0735; A61P 25/28; A61K 31/7088; A61K 31/00; C07K 16/28

(52) U.S. Cl. CPC A61K 35/545; C12N 5/0606; A61P 25/28; A61K 31/7088; A61K 31/00; C07K 16/28

(57) ABSTRACT

Materials and methods for modulating protease activated receptor 2 (PAR2) activity in order to alter myelination or demyelination are provided herein.

### CROSS-REFERENCE TO RELATED APPLICATIONS

This application is divisional of U.S. application Ser. No. 16/312,411, filed Dec. 21, 2018, which is a National Stage application under 35 U.S.C. § 371 of International Application No. PCT/US2017/038971, having an International Filing Date of Jun. 23, 2017, which claims benefit of priority from U.S. Provisional Application No. 62/353,769, filed Jun. 23, 2016.

### STATEMENT AS TO FEDERALLY SPONSORED RESEARCH

This invention was made with government support under NS052741 awarded by the National Institutes of Health. The government has certain rights in the invention.

### TECHNICAL FIELD

This document relates to materials and methods for modulating protease activated receptor 2 (PAR2) activity to alter myelination.

### BACKGROUND

Myelination in the central nervous system is achieved through a delicate balance of extrinsic and intrinsic signaling mechanisms. Myelin not only enhances axonal conduction velocity, but also provides protection and trophic support (Wilkins et al., J Neurosci, 23(12):4967-4974, 2003). Normal myelination requires a series of well-orchestrated events, including the generation of oligodendrocyte progenitors (OPCs), migration of the OPCs to specific regions of the brain or spinal cord, and differentiation of the OPCs into oligodendrocytes that elaborate multilamellar sheaths of plasma membrane to myelinate axons in precise relation to their diameter. Aberrations in this process during the perinatal period can result in white matter injury and profound sensorimotor and cognitive disabilities. Multiple factors can disrupt the key developmental mileposts, including hemorrhagic-ischemic injuries (Mifsud et al., CNS Neurosci Ther 20:603-612, 2014; Crawford et al., J Comp Pathol 149:242-254, 2013; and Volpe et al., Int J Devel Neurosci 29:423-440, 2011).

### SUMMARY

This document is based, at least in part, on elucidation of the role of PAR2 in regulating myelination and demyelination, and the development of methods for targeting PAR2 to increase myelination and locomotor activity in vivo. As demonstrated by the data presented herein, PAR2 is a therapeutic target for increasing myelination in the developing and adult central nervous system (CNS). For example, the methods disclosed herein can be used to prevent perinatal white matter injuries, and provide opportunities to improve both short and long term neurological functional outcomes. Collectively, the data described herein identify PAR2 as an innate suppressor of developmental spinal cord myelination, and of neural stem cell expansion and myelin regeneration in the adult CNS. Thus, PAR2 is identified as a target for therapies aimed at promoting myelinogenesis, myelin preservation, and myelin regeneration in neurological conditions that can affect the developing and/or developed (e.g., adult) CNS in which white matter injury and repair is a central concern.

In one aspect, this document features a method for increasing myelination or remyelination in a mammal. The method can include (a) identifying the mammal as being in need of increased myelination or remyelination, and (b) administering to the mammal an agent that reduces the activity of PAR2, or a composition containing an agent that reduces the activity of PAR2, wherein the agent is administered in an amount effective to increase myelination or remyelination in the mammal. The agent can be a small molecule inhibitor of PAR2, an antibody against PAR2, an inhibitory RNA, or an antisense nucleic acid molecule. The mammal can be a human (e.g., a preterm infant, an infant, a child, a teenager, or an adult). The mammal can be identified as having a CNS demyelinating condition. The CNS demyelinating condition can be a CNS injury, multiple sclerosis (MS), amyotrophic lateral sclerosis (ALS), Alzheimer's disease (AD), a spinal cord injury, a neuropsychiatric disorder, or stroke.

In another aspect, this document features a method for promoting myelin protection or preservation in a mammal. The method can include administering to the mammal an agent that reduces the activity of PAR2, or a composition containing an agent that reduces the activity of PAR2, wherein the agent is administered in an amount effective to reduce or prevent demyelination in the mammal. The agent can be a small molecule inhibitor of PAR2, an antibody against PAR2, an inhibitory RNA, or an antisense nucleic acid molecule. The mammal can be a human (e.g., a preterm infant, an infant, a child, a teenager, or an adult). The mammal can be identified as having a CNS demyelinating condition, such as a CNS injury, MS, ALS, AD, a spinal cord injury, a neuropsychiatric disorder, or stroke.

In another aspect, this document features a method for promoting differentiation of an OPC. The method can include contacting the OPC with an agent that reduces the activity of PAR2, or a composition containing an agent that reduces the activity of PAR2. The agent can be a small molecule inhibitor of PAR2, an antibody against PAR2, an inhibitory RNA, or an antisense nucleic acid molecule. The contacting can be in vivo, such as in a mammal (e.g., a mammal identified as having a CNS demyelinating condition such as a CNS injury, MS, ALS, AD, a spinal cord injury, a neuropsychiatric disorder, or stroke).

In still another aspect, this document features a method for promoting expansion of a population of neural stem cells. The method can include contacting the population with an agent that reduces the activity of PAR2, or a composition containing an agent that reduces the activity of PAR2. The agent can be a small molecule inhibitor of PAR2, an antibody against PAR2, an inhibitory RNA, or an antisense nucleic acid molecule. The contacting can be in vivo. The contacting can occur in a mammal (e.g., a human). The mammal can be identified as having a CNS demyelinating condition, such as a CNS injury, MS, ALS, AD, a spinal cord injury, a neuropsychiatric disorder, or stroke.

In addition, this document features a method for promoting differentiation of a population of neural stem cells toward myelinating cells. The method can include contacting the population with an agent that reduces the activity of PAR2, or a composition containing an agent that reduces the activity of PAR2. The agent can be a small molecule inhibitor of PAR2, an antibody against PAR2, an inhibitory RNA, or an antisense nucleic acid molecule. The contacting can be in vivo. The contacting can occur in a mammal (e.g., a human). The mammal can be identified as having a CNS demyelinating condition, such as a CNS injury, MS, ALS, AD, a spinal cord injury, a neuropsychiatric disorder, or stroke.

This document also features a method for promoting generation of oligodendrocytes in a mammal. The method can include (a) identifying the mammal as being in need of increased numbers of oligodendrocytes, and (b) administering to the mammal an agent that reduces the activity of PAR2, or a composition containing an agent that reduces the activity of PAR2. The agent can be a small molecule inhibitor of PAR2, an antibody against PAR2, an inhibitory RNA, or an antisense nucleic acid molecule. The mammal can be a human (e.g., a preterm infant, an infant, a child, a teenager, or an adult). The mammal can be identified as having a CNS demyelinating condition (e.g., a CNS injury, MS, ALS, AD, a spinal cord injury, a neuropsychiatric disorder, or stroke). In yet another aspect, this document features a method for treating a CNS demyelinating condition in a mammal. The method can include administering to the mammal an agent that reduces the activity of PAR2, or a composition containing an agent that reduces the activity of PAR2, wherein the composition is administered in an amount effective to reduce or prevent demyelination, or to enhance myelination or remyelination. The agent can be a small molecule inhibitor of PAR2, an antibody against PAR2, an inhibitory RNA, or an antisense nucleic acid molecule. The mammal can be a human (e.g., a preterm infant, a child, a teenager, or an adult). The mammal can be identified as having a CNS demyelinating condition, such as a CNS injury, MS, ALS, AD, a spinal cord injury, a neuropsychiatric disorder, or stroke. In another aspect, this document features a method for increasing myelination or remyelination in a subject. The method can include delivering to the subject a plurality of modified stem cells that have reduced PAR2 expression as compared to corresponding wild type stem cells. The modified stem cells can be neural stem cells having a mutation in the PAR2 gene. The subject can be a human (e.g., a preterm infant, a child, a teenager, or an adult). The subject can be identified as having CNS demyelinating condition, such as a CNS injury, MS, ALS, AD, a spinal cord injury, a neuropsychiatric disorder, or stroke.

In another aspect, this document features a method for treating a CNS demyelinating condition in a mammal in need thereof. The method can include administering to the mammal a composition containing a plurality of modified stem cells that have reduced PAR2 expression as compared to corresponding wild type stem cells, wherein the composition is administered in an amount effective to reduce or prevent demyelination, or to enhance myelination or remyelination. The modified stem cells can be neural stem cells having a mutation in the PAR2 gene. The CNS demyelinating condition can be a CNS injury, MS, ALS, AD, a spinal cord injury, a neuropsychiatric disorder, or stroke. The mammal can be a human (e.g., a preterm infant, a child, a teenager, or an adult).

In addition, this document features compositions for use in increasing myelination, increasing remyelination, promoting myelin protection, or promoting myelin preservation in a mammal (e.g., a human) in need thereof, where the compositions contain an agent that reduces the activity of PAR2.

This document also features compositions for increasing myelination or remyelination in a subject, or for treating a CNS demyelinating condition in a mammal in need thereof, where the compositions contain a plurality of modified stem cells that have reduced PAR2 expression as compared to corresponding wild type stem cells. The compositions can contain an amount of modified stem cells that is effective to reduce or prevent demyelination, or to enhance myelination or remyelination, when administered to a mammal in need thereof.

In another aspect, this document features the use of an agent that reduces the activity of PAR2 in the manufacture of a medicament for increasing myelination, increasing remyelination, promoting myelin protection, or promoting myelin preservation in a mammal, for promoting differentiation of oligodendrocyte precursor cells (OPCs), for promoting expansion of a population of neural stem cells, for promoting generation of oligodendrocytes in a mammal, for treating a CNS demyelinating condition in a mammal, or for promoting differentiation of a population of neural stem cells toward myelinating cells.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1N show that enhanced expression of myelin-associated proteins and promyelination signaling occurs in the spinal cord of mice with PAR2-loss-of function. Western blots of whole spinal cord homogenates and associated histograms (FIGS. 1A to 1G) illustrate that mice lacking PAR2 show significant increases in the expression of PLP at P0 (FIG. 1B) and MBP by P45 (FIG. 1C). Higher levels of Olig2 protein occurred in the PAR2−/− spinal cord on P7 compared to wild type. Increases in the pro-myelination signaling pathway ERK1/2 also were observed by P7, on P21, and in adulthood (FIGS. 1H and 1I). No significant differences in NFH (FIG. 1F) or NFL (FIG. 1G) were observed in the same spinal cord samples. Levels of total AKT were significantly elevated in PAR2−/− mice on P7 (FIG. 1N). ROD readings for pERK and pAKT were normalized to the total protein or to Actin (FIGS. 1I to 1K). Actin was probed on every membrane to control for loading, and is shown for the corresponding membrane in the lower panel in FIG. 1A and FIG. 1H. *P<0.05, **P≤0.01, ***P≤0.001, Newman Keuls; ND, not detected.

FIG. 2 shows that PAR2 loss-of-function results in accelerated generation and differentiation of oligodendrocytes. At P7, the number of Olig2- and CC-1-immunopositive cells within the dorsal column of the spinal cord was increased by 1.4- to 1.5-fold in mice with PAR2 loss-of-function (P≤0.02, Newman Keuls). Parallel elevations in Olig2 protein were seen by Western blot (see FIGS. 1A and 1E). *P<0.05, **P≤0.01, ***P≤0.001 Newman Keuls; N D, not detected. Scale bar B and D=30 μm.

FIGS. 3A-3D show that oligodendrocyte progenitor cells with genetic or pharmacologic PAR2 loss-of-function exhibit increased expression of myelin-associated proteins in vitro. Photomicrographs and associated histograms (FIGS. 3A and 3B) show that PAR2−/− OPCs differentiated for 72 hours had a greater number of PLP-immunoreactive cells, and more PLP per cell, compared to PAR2+/+ cells. *P=0.01, Students t-test, Scale bar=20 μm. Similar changes were seen at the RNA level (FIGS. 3C and 3D) in OPCs lacking the PAR2 gene (FIG. 3C), or in OPCs that were treated with a PAR2 small molecule inhibitor (5 μM GB88; FIG. 3D), including a significant increase in PLP, MBP and NogoA expression (***P=0.001, Students t-test). No significant changes were seen Olig2 RNA expression under the same conditions.

FIGS. 4A-4D show that PAR2-loss-of-function enhances myelin thickness in the adult spinal cord. FIGS. 4A and 4B are representative electron micrographs taken from the spinal cord dorsal column white matter of PAR2+/+ or PAR2−/− mice at P45. Micrographs were used to calculate g-ratios and myelin thickness, which were plotted relative to axon diameter (FIG. 4C). At P45, mean g-ratios were significantly lower in PAR2−/− mice (0.72±0.002) compared to PAR2+/+ mice (0.75±0.002, P=0.37×10⁻⁴³, Students t-test) and myelin thickness was significantly greater (PAR2−/−=0.29±0.003 μm; PAR2+/+=0.28±0.003 μm; P=0.01, Students t-test). The graphs in FIG. 4D show the mean g-ratios and myelin thickness for axons across a range of diameters, demonstrating that the most significant increases in myelin thickness were observed in axons ranging from 0.5 to 1 μm (P≤1.5×10⁻⁶, Students t-test). Scale bar, A=2 μm; B=0.2 μm).

FIGS. 5A-5D show that PAR2 loss-of-function results in an increase in the number of oligodendrocytes in the adult brain. The number of Olig2- or CC-1-immunoreactive cells in the anterior commissure of 8 week old adult mice was greater in mice with PAR2 genetic deletion (FIGS. 5A, right panel, and 5B, lower panels) compared to wild type mice (FIGS. 5A, left panel, and 5B, upper panels). The number of Olig2-immunoreactive cells was also greater in the corpus callosum of mice with PAR2 loss-of-function compared to wild type mice (FIGS. 5C, left panel, and 5D, left panels). The number of CC-1-immunoreactive cells was not significantly different in the corpus callosum of mice with PAR2 loss-of-function as compared to wild type mice (FIGS. 5C, right panel, and 5D, right panels). Sections were counterstained with methyl green such that nuclei appeared green, while immunostained Olig2 or CC-1 cells were brown. *P<0.05, ***P<0.001, Students t-test. Scale bar=50 μm.

FIGS. 6A-6D show that PAR2 loss-of-function results in an increase in the number of neural stem cells and oligodendrocyte progenitor cells present in the subventricular zone (SVZ) of the adult brain. Counts of Sox2+ multipotent neural stem cells were greater in the SVZ of PAR2−/− compared to PAR2−/− adult mice (FIGS. 6C and 6D, top panels). A greater number of cells positive for the proliferation marker Ki-67 were also observed in the SVZ of mice with PAR2 loss-of-function (FIGS. 6C and 6D, bottom panels). Counts were made in coronal sections taken +0.5 mm from Bregma (FIGS. 6A and 6B). All sections were counterstained with methyl green such that nuclei appeared green and immunostained cells appeared brown. *P<0.05; ***P<0.001, Students t-test. Scale bar=40 μm.

FIGS. 7A-7C show that PAR2 gene loss-of-function results in increased nocturnal locomotor activity. A comprehensive laboratory animal monitoring system was used to demonstrate that PAR2−/− mice have (FIG. 7A) higher total nocturnal activity under fed (P=0.04) or fasted (P=0.001) conditions, (FIG. 7B) higher nocturnal ambulation under fed (P=0.03) or fasted (P=0.0009) night conditions, and (FIG. 7C) higher rearing under fed (P=0.05) or fasted (P=0.002) night conditions (Students unpaired t-test). No significant differences were observed in daytime activity, ambulation, or rearing under any conditions.

FIGS. 8A-8F show that mice lacking PAR2 show improvements in the abundance of myelin and myelinating cells after traumatic spinal cord injury. FIG. 8A is a Western blot and FIG. 8B is an associated histogram demonstrating higher levels of myelin basic protein (MBP) in the spinal cord of mice with PAR2 loss-of-function at base line, and at 3 and 30 dpi. MBP levels were higher in spinal segments at the injury epicenter and above at 3 dpi, and in spinal segments above at 30 dpi in PAR2−/− relative to those with an intact PAR2 signaling system. Photomicrographs and associated histograms (FIGS. 8C to 8F) demonstrate that the number of Olig2+- and CC-1+-oligodendrocytes was higher in the spinal cord of PAR2−/− compared to PAR2+/+ mice at baseline. A greater number of Olig2+-oligodendrocytes also were observed in spinal segments above the injury epicenter at 30 dpi in mice with PAR2 loss-of-function. Scale bars in FIGS. 8C and 8E=50 μm. C, control; E, injury epicenter; A, above epicenter; B, below epicenter.

FIGS. 9A-9C show that myelin regeneration was enhanced in mice with PAR2 loss-of-function. The photomicrographs in FIGS. 9A and 9B show examples of paraphenylenediamine (PPD) stained myelin sheaths in the spinal cord dorsal column white matter of control PAR2+/+ or PAR2−/− mice and in those at 14 days after injection of the demyelinating agent lysophosphatidyl choline. Counts of remyelinated axons at 14 days demonstrated 25% more remyelinated axons per mm² in mice with PAR2 loss-of-function (see also FIG. 9C; P=0.04, Students t-test). Remyelinated axons are identified as those with thinner and more lightly PPD stained myelin sheaths. The boxed areas through the 14 day remyelinated lesions are provided at higher magnification to facilitate visualization of remyelinated axons. Scale bar B=20 μm and insert=10 μm.

### DETAILED DESCRIPTION

Demyelination also can occur as a result of injury to the brain or spinal cord. Demyelinating disease in the CNS causes deterioration of the myelin sheaths that cover nerve cells in the brain, spinal cord, and optic nerve, preventing the nerves from properly transmitting impulses. Demyelination also can occur in the peripheral nerves.

CNS demyelinating diseases include, for example, multiple sclerosis (MS), which is the most common demyelinating disease of the CNS. A number of demyelinating diseases, such as optic neuritis, neuromyelitis optica, and Leber's hereditary optic neuropathy, affect the optic nerve. Other CNS demyelinating diseases include amyotrophic lateral sclerosis (ALS), Alzheimer's disease (AD), Tay-Sachs disease, adrenoleuko-dystrophy, adrenomyeloneuropathy, and transverse myelitis. Demyelination also can be caused by autoimmune disease, infection, nutritional deficiencies, and low oxygen levels.

The symptoms of CNS demyelinating diseases can affect any part of the CNS, and may include seizures, headaches, delirium, confusion, and/or slurred speech. In some cases, muscle weakness, paralysis, trouble with balance, difficulty walking, tremors, pain, numbness, tingling affect some with the disease, vision and hearing problems, and/or bladder problems can occur. Demyelination disorders tend to progress over time, and some forms of CNS demyelination can lead to early death or disability. For example, while people with MS often have a normal or near-normal life expectancy, hereditary demyelination disorders such Tay-Sachs disease can end in early death.

PAR2 [also referred to as coagulation factor II (thrombin) receptor-like 1 (F2RL1) or G-protein coupled receptor 11 (GPR11)] is a protein that, in humans, is encoded by the F2RL1 gene. PAR2 modulates inflammatory responses and acts as a sensor for proteolytic enzymes generated during infection (Lee et al., Yonsei Med J 51(6):808-822, 2010). PAR2 is a member of the protease-activated receptor family, and also is a member of a large family of 7-transmembrane receptors that couple to guanosine-nucleotide-binding proteins. PAR2 is activated by trypsin (but, unlike PAR1, not by thrombin), by proteolytic cleavage of its extracellular N-terminal domain. The new amino terminus resulting from cleavage functions as a tethered ligand, and activates the receptor. PAR2 also can be activated non-proteolytically by exogenous peptide sequences that mimic the final amino acids of the tethered ligand. PAR2 is positioned to serve as a key translator of the proteolytic microenvironment in the developing, adult and injured central nervous system into cellular responses that regulate myelin homeostasis and regeneration.

Oligodendrocytes are essential regulators of energy homeostasis, and produce the myelin that insulates neural axons, thereby facilitating electrical conduction in the developing and adult central nervous system. Oligodendrocytes therefore are a target for the design of therapies to promote recovery of function in cases of injury and disease.

As described in the Examples herein, a murine genetic model was used to functionally evaluate the role of PAR2 in the process of murine spinal cord myelination at cellular, molecular, and ultrastructural levels. The experimental results demonstrated that PAR2 is a suppressor of developmental myelination, and that its absence results in increased expression of PLP and Olig2, increased ERK1/2 signaling, and increased oligodendrocyte numbers in the perinatal spinal cord, enhanced expression of myelin proteins in differentiated oligodendroglia in vitro, and higher levels of MBP, thicker myelin sheaths, increased numbers of oligodendrocytes and neural stem cells, and enhanced motor activity in adulthood. The experiments described herein also showed that loss of PAR2 can improve myelin integrity after spinal cord injury, and can facilitate myelin regeneration in the adult spinal cord.

This document therefore provides materials and methods for modulating myelination and enhancing oligodendrocyte and neural stem cell numbers in a subject, by delivering to the subject an agent that reduces the activity of PAR2. The subject can be, for example, a mammal, such as a mouse, rat, rabbit, dog, cat, monkey, or human, including preterm infants as well as juveniles or adults who are in need of increased myelination. Since PAR2 acts to suppress myelination, reducing PAR2 activity can increase myelination. In some embodiments, therefore, a subject identified as having or as being at risk for having a CNS demyelinating disorder can be given an agent that reduces the level of PAR2 activity. In some cases, an agent can inhibit the action of the PAR2 protein, while in other cases an agent can inhibit expression of the PAR2 gene.

Suitable agents include, for example, small molecules, antibodies or antibody fragments, such as Fab′ fragments, F(ab′) 2 fragments, or scFv fragments that bind PAR2, antisense oligonucleotides, interfering RNA (RNAi, including short interfering RNA (siRNA) and short hairpin RNA (shRNA)), or combinations thereof.

Useful small molecule inhibitors of PAR2 may include, for example, Pepducins such as P2pal-18S (Sevigney et al., Proc Natl Acad Sci USA 108:8491-8496, 2011; and Yoon et al., J Neurochem 127:283-298, 2013); small molecule inhibitors such as GB88 (Lohman et al., FASEB J26(7):2877-2887, 2012; Lohman et al., Pharmacol Exp Ther 340:256-265, 2012; and Suen et al., Brit J Pharmacol 171:4112-4124, 2012); neutralizing antibodies (Mandal et al., Blood 110:161-170, 2007), and siRNA approaches.

Methods for producing antibodies and antibody fragments are known in the art. Chimeric antibodies and humanized antibodies made from non-human (e.g., mouse, rat, gerbil, or hamster) antibodies also can be useful. Chimeric and humanized monoclonal antibodies can be produced by recombinant DNA techniques known in the art, for example, using methods described in U.S. Pat. Nos. 4,816,567; 5,482,856; 5,565,332; 6,054,297; and 6,808,901.

Antisense oligonucleotides as provided herein are at least 8 nucleotides in length and hybridize to a PAR2 transcript. For example, a nucleic acid can be about 8, 9, 10 to 20 (e.g., 11, 12, 13, 14, 15, 16, 17, 18, 19, or 20 nucleotides in length), 15 to 20, 18 to 25, or 20 to 50 nucleotides in length. In some embodiments, antisense molecules greater than 50 nucleotides in length can be used, including the full-length sequence of a PAR2 mRNA. As used herein, the term “oligonucleotide” refers to an oligomer or polymer of ribonucleic acid (RNA) or deoxyribonucleic acid (DNA) or analogs thereof. Nucleic acid analogs can be modified at the base moiety, sugar moiety, or phosphate backbone to improve, for example, stability, hybridization, or solubility of a nucleic acid. Modifications at the base moiety include substitution of deoxyuridine for deoxythymidine, and 5-methyl-2′-deoxycytidine and 5-bromo-2′-deoxycytidine for deoxycytidine. Other examples of nucleobases that can be substituted for a natural base include 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Other useful nucleobases include those disclosed, for example, in U.S. Pat. No. 3,687,808.

Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six-membered, morpholino ring, or peptide nucleic acids, in which the deoxyphosphate backbone is replaced by a pseudopeptide backbone (e.g., an aminoethylglycine backbone) and the four bases are retained. See, for example, Summerton and Weller, Antisense Nucleic Acid Drug Dev. 7:187-195, 1997; and Hyrup et al., Bioorgan. Med. Chem. 4:5-23, 1996. In addition, the deoxyphosphate backbone can be replaced with, for example, a phosphorothioate or phosphorodithioate backbone, a phosphoroamidite, or an alkyl phosphotriester backbone. See, for example, U.S. Pat. Nos. 4,469,863; 5,235,033; 5,750,666; and 5,596,086 for methods of preparing oligonucleotides with modified backbones.

Antisense oligonucleotides also can be modified by chemical linkage to one or more moieties or conjugates that enhance the activity, cellular distribution or cellular uptake of the oligonucleotide. Such moieties include but are not limited to lipid moieties (e.g., a cholesterol moiety); cholic acid; a thioether moiety (e.g., hexyl-S-tritylthiol); a thiocholesterol moiety; an aliphatic chain (e.g., dodecandiol or undecyl residues); a phospholipid moiety (e.g., di-hexadecyl-rac-glycerol or triethyl-ammonium 1,2-di-O-hexadecyl-rac-glycero-3-H-phosphonate); a polyamine or a polyethylene glycol chain; adamantane acetic acid; a palmityl moiety; or an octadecylamine or hexylamino-carbonyl-oxycholesterol moiety. The preparation of such oligonucleotide conjugates is disclosed in, for example, U.S. Pat. Nos. 5,218,105 and 5,214,136.

Methods for synthesizing antisense oligonucleotides are known, including solid phase synthesis techniques. Equipment for such synthesis is commercially available from several vendors including, for example, Applied Biosystems (Foster City, Calif.).

Alternatively, expression vectors that contain a regulatory element that directs production of an anti sense transcript can be used to produce antisense molecules.

Antisense oligonucleotides can bind to a nucleic acid encoding PAR2, including DNA encoding PAR2 RNA (including pre-mRNA and mRNA) transcribed from such DNA, and also cDNA derived from such RNA, under physiological conditions (i.e., physiological pH and ionic strength).

It is understood in the art that the sequence of an antisense oligonucleotide need not be 100% complementary to that of its target nucleic acid to be hybridizable under physiological conditions. Antisense oligonucleotides hybridize under physiological conditions when binding of the oligonucleotide to the PAR2 nucleic acid interferes with the normal function of the PAR2 nucleic acid, and non-specific binding to non-target sequences is minimal.

Target sites for PAR2 antisense oligonucleotides can include the regions encompassing the translation initiation or termination codon of the open reading frame (ORF) of the gene. In addition, the ORF can be targeted effectively in antisense technology, as have the 5′ and 3′ untranslated regions. In some embodiments, antisense oligonucleotides can be directed at intron regions and intron-exon junction regions. Further criteria can be applied to the design of antisense oligonucleotides. Such criteria are well known in the art, and are widely used, for example, in the design of oligonucleotide primers. These criteria include the lack of predicted secondary structure of a potential antisense oligonucleotide, an appropriate G and C nucleotide content (e.g., approximately 50%), and the absence of sequence motifs such as single nucleotide repeats (e.g., GGGG runs). The effectiveness of antisense oligonucleotides at modulating expression of a PAR2 nucleic acid can be evaluated by measuring levels of the PAR2 mRNA or polypeptide (e.g., by Northern blotting, RT-PCR, Western blotting, ELISA, or immunohistochemical staining).

Single and double-stranded interfering RNA (RNAi, such as siRNA and shRNA) homologous to PAR2 DNA also can be used to reduce expression of PAR2 and consequently, activity of PAR2. Methods for using interfering RNA technology in different species are known in the art. See, e.g., U.S. Pat. No. 6, 933,146; Fire et al., Nature 391:806-811, 1998; Romano and Masino, Mol. Microbiol. 6:3343-3353, 1992; Cogoni et al., EMBO J. 15:3153-3163, 1996; Cogoni and Masino, Nature 399:166-169, 1999; Misquitta and Paterson, Proc. Natl. Acad. Sci. USA 96:1451-1456, 1999; and Kennerdell and Carthew, Cell 95:1017-1026, 1998.

The sense and anti-sense RNA strands of RNAi can be individually constructed using chemical synthesis and enzymatic ligation reactions using procedures known in the art. For example, each strand can be chemically synthesized using naturally occurring nucleotides or nucleic acid analogs. The sense or anti-sense strand also can be produced biologically using an expression vector into which a target PAR2 sequence (full-length or a fragment) has been subcloned in a sense or anti-sense orientation. The sense and anti-sense RNA strands can be annealed in vitro before delivery of the dsRNA to cells. Alternatively, annealing can occur in vivo after the sense and anti-sense strands are sequentially delivered to the tumor vasculature or to tumor cells.

One or more agents that modulate PAR2 levels or activity can be incorporated into a pharmaceutical composition, such as by combination with a pharmaceutically acceptable carrier. Pharmaceutically acceptable carriers are biologically compatible vehicles that are suitable for administration to a mammal (e.g., a human), and include, for example, water, physiological saline, and liposomes. Pharmaceutically acceptable carriers can be selected with the planned manner of administration in mind so as to provide for the desired bulk, consistency, and other pertinent transport and chemical properties, when combined with one or more of components of a given pharmaceutical composition. For example, one or more agents can be formulated for delivery orally or by intravenous infusion, or injected subcutaneously, intramuscularly, intrathecally, intraperitoneally, intrarectally, intravaginally, intranasally, intragastrically, intratracheally, or intrapulmonarily. In some embodiments, one or more agents can be delivered directly to the CNS by, for example, injection or infusion into the cerebrospinal fluid, optionally with one or more additional agents that are capable of promoting penetration of the first agent across the blood-brain barrier.

The dosage for any one patient required depends on may factors, including the particular agent(s) being administered, time and route of administration, the nature of the formulation, the nature of the patient's illness, the subject's size, weight, surface area, age, and gender, other drugs being administered concurrently, and the judgment of the attending clinician. Suitable dosages typically are in the range of about 10 ng/kg body weight to about 1 g/kg body weight, although wide variations in the needed dosage are to be expected in view of the variety of agents available and the differing efficiencies of various routes of administration. For nucleic acids, dosages may range from about 10⁶ to about 10¹² copies of the nucleic acid. Further, dosages can be administered on a repeated basis (e.g., on a daily, weekly, or monthly basis, such as once a day, every other day, twice weekly, weekly, twice monthly, or monthly). Variations in dosage levels can be adjusted using standard empirical routines for optimization as is well understood in the art. Encapsulation of an agent in a suitable delivery vehicle (e.g., polymeric microparticles or an implantable device) may increase the efficiency of delivery, particularly for oral delivery.

In some embodiments, a nucleic acid (e.g., an expression vector containing a regulatory sequence operably linked to a nucleic acid encoding an antisense oligonucleotide, or an expression vector from which sense and anti-sense RNAs can be transcribed under the direction of separate promoters, or a single RNA molecule containing both sense and anti-sense sequences can be transcribed under the direction of a single promoter) can be delivered to appropriate cells in a subject. Suitable expression vectors include, for example, plasmids and viral vectors such as herpes viruses, retroviruses, vaccinia viruses, attenuated vaccinia viruses, canary pox viruses, adenoviruses and adeno-associated viruses, among others.

Expression of a nucleic acid can be directed to any cell in the body of the subject. However, it can be particularly useful to direct expression to cells in, or close to, the CNS. Targeted expression can be achieved by, for example, the use of polymeric, biodegradable microparticle or microcapsule delivery devices known in the art and/or tissue or cell-specific antibodies. Alternatively, tissue specific targeting can be achieved by the use of tissue-specific transcriptional regulatory sequences (i.e., tissue specific promoters) which are known in the art.

Nucleic acids also can be delivered to cells using liposomes, which can be prepared by standard methods. Vectors can be incorporated alone into these delivery vehicles, or can be co-incorporated with tissue-specific antibodies. Alternatively, a molecular conjugate composed of a plasmid or other vector attached to poly-L-lysine by electrostatic or covalent forces can be prepared. Poly-L-lysine binds to a ligand that can bind to a receptor on target cells (Cristiano et al., J. Mol. Med. 73:479, 1995). Delivery of “naked DNA” (i.e., without a delivery vehicle) to an intramuscular, intradermal, or subcutaneous site is another means to achieve in vivo expression.

In addition, a method can be an ex vivo procedure that involves providing a recombinant cell that is, or is a progeny of a cell, obtained from a subject and has been transfected or transformed ex vivo with one or more nucleic acids encoding one or more agents that reduce PAR2 activity (e.g., an siRNA targeted to PAR2), so that the cell expresses the agent(s); and administering the cell to the subject. The cells can be cells obtained from the subject to whom they are to be administered, or from another subject. The donor and recipient of the cells can have identical major histocompatibility complex (MHC; HLA in humans) haplotypes. In some embodiments, the donor and recipient are homozygotic twins or are the same individual (i.e., are autologous). The recombinant cells can also be administered to recipients that have no, or only one, two, three, or four MHC molecules in common with the recombinant cells, e.g., in situations where the recipient is severely immuno-compromised, where only mismatched cells are available, and/or where only short term survival of the recombinant cells is required or desirable.

The efficacy of an agent can be evaluated both in vitro and in vivo. Briefly, an agent can be tested and/or used for its ability to, for example, (a) reduce PAR2 activity, (b) increase myelination, (c) inhibit or slow the progression of demyelination, or (d) promote differentiation of OPCs or generation of oligodendrocytes. For in vivo methods, the agent can, for example, be injected into an animal (e.g., a mouse model of CNS demyelination), and its effects then can be assessed. Suitable methods for evaluating the level or progression of myelination/demyelination include, without limitation, imaging, motor evoked potential, visual evoked potentials, sensorimotor, and cognitive functional outcomes. Based on the results, an appropriate dosage range and administration route can be determined. For in vitro or ex vivo methods, a population of cells (e.g., OPCs) can be contacted with the agent.

This document provides methods for increasing myelination or remyelination in a subject, promoting myelin protection or preservation in a subject, promoting generation of oligodendrocytes or differentiation of OPCs in a subject, promoting expansion of neural stem cells in a subject, and/or promoting differentiation of neural stem cells toward myelinating cells in a subject. In some embodiments, the methods provided herein can include identifying a subject as being in need of increased myelination, or in need of increased oligodendrocyte numbers. In some embodiments, the subject can be identified on the basis of, for example, having a disorder characterized by demyelination (e.g., demyelination in the CNS). The subject can be a mammal (e.g., a human, including preterm infant, a child, a teenager, or an adult, or a non-human mammal). In some cases, the subject can be identified as having a neuroinflammatory disease (e.g., MS or AD), ALS, a stroke, or an injury to the CNS (e.g., the spinal cord). Alternatively, a subject can be identified as being in need of increased myelination but not having an inflammatory condition, or not having an inflammatory condition in the CNS.

In some embodiments of the methods provided herein, one or more agents that reduce PAR2 activity, or a composition containing one or more such agents, can be administered to a subject in an amount effective to treat a CNS demyelinating disorder, to reduce or prevent demyelination, to enhance myelination or remyelination, to promote differentiation of OPCs and generation of oligodendrocytes, to promote expansion of neural stem cells, and/or to promote differentiation of neural stem cells toward myelinating cells.

For example, an effective amount of a PAR2-modulating agent or a composition containing one or more PAR2-modulating agents can reduce the level or rate of demyelination in a subject by at least 10 percent (e.g., at least 20, 30, 40, 50, 60, 70, 80, 90, 10 to 25, 25 to 50, 50 to 75, or 75 to 100 percent) as compared to the level or rate of demyelination in the subject prior to treatment, or as compared to the level or rate of demyelination in an untreated subject. In some embodiments, an effective amount of a PAR2-modulating agent or a composition containing one or more PAR2-modulating agents can increase the level or rate of myelination or remyelination in a subject by at least 10 percent (e.g., at least 20, 30, 40, 50, 60, 70, 80, 90, 10 to 25, 25 to 50, 50 to 75, 75 to 100, or more than 100 percent) as compared to the level or rate of remyelination in the subject prior to treatment, or as compared to the level or rate of remyelination in an untreated subject. Further, an effective amount of a PAR2-modulating agent or a composition containing one or more PAR2-modulating agents can increase the number of OPCs, oligodendrocytes, and/or neural stem cells in a subject (or in a biological sample from a subject) by at least 10 percent (e.g., at least 20, 30, 40, 50, 60, 70, 80, 90, 10 to 25, 25 to 50, 50 to 75, 75 to 100, or more than 100 percent) as compared to the number of OPCs, oligodendrocytes, and/or neural stem cells in an untreated subject.

The therapeutically effective amount of a PAR2-modulating agent can be dependent on the particular agent that is utilized, the subject being treated, the severity and type of the affliction, and the manner of administration. For example, a therapeutically effective amount of a PAR2-modulating agent can range from about 0.01 μg per kg body weight to about 1 g per kg body weight (e.g., about 0.1 μg/kg to about 1 μg/kg, about 1 μg/kg to about 5 μg/kg, about 5 μg/kg to about 100 μg/kg, about 100 μg/kg to about 500 μg/kg, about 500 μg/kg to about 1 mg/kg, about 1 mg/kg to about 100 mg/kg, about 100 mg/kg to about 500 mg/kg, or about 500 mg/kg to about 1 g/kg body weight). The exact dose can be readily determined by those of skill in the art, based on the potency of the specific compound the age, weight, sex and physiological condition of the subject. In addition, single or multiple administrations of a PAR2-modulating agent can be given depending on the dosage and frequency as required and tolerated by the subject. In some embodiments, the dosage is administered once, but in other embodiments the dosage can be administered periodically (e.g., until a therapeutic result is achieved). Generally, the dose is sufficient to treat or ameliorate symptoms or signs of disease without producing unacceptable toxicity to the subject.

In some cases, a method as provided herein can include delivering to a subject a population of stem cells that have been modified to have reduced PAR expression as compared to corresponding wild type neural stem cells. For example, the stem cells can be modified in vitro to contain a mutation in the PAR2 gene, such that PAR2 expression is reduced or even knocked out. Suitable types of stem cells include, without limitation, embryonic stem cells, induced pluripotent stem cells, bone marrow derived stem cells, mesenchymal stem cells, and neural stem cells. After delivery to the subject (e.g., a preterm infant, or a juvenile or adult having a CNS injury or demyelinating disorder), the stem cells can differentiate into neuronal cells and, due to their reduced level of PAR2 expression, can facilitate or enhance myelination.

The invention will be further described in the following examples, which do not limit the scope of the invention described in the claims.

### EXAMPLES

### Example 1

### PAR2 Loss-of-Function Accelerates the Expression of PLP and Olig2 in the Perinatal Spinal Cord and Results in Higher MBP Levels in Adulthood

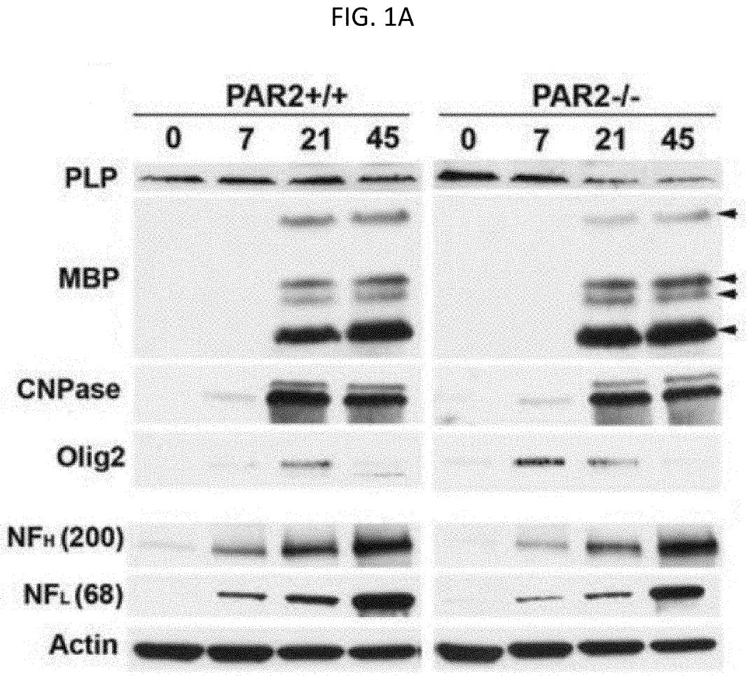

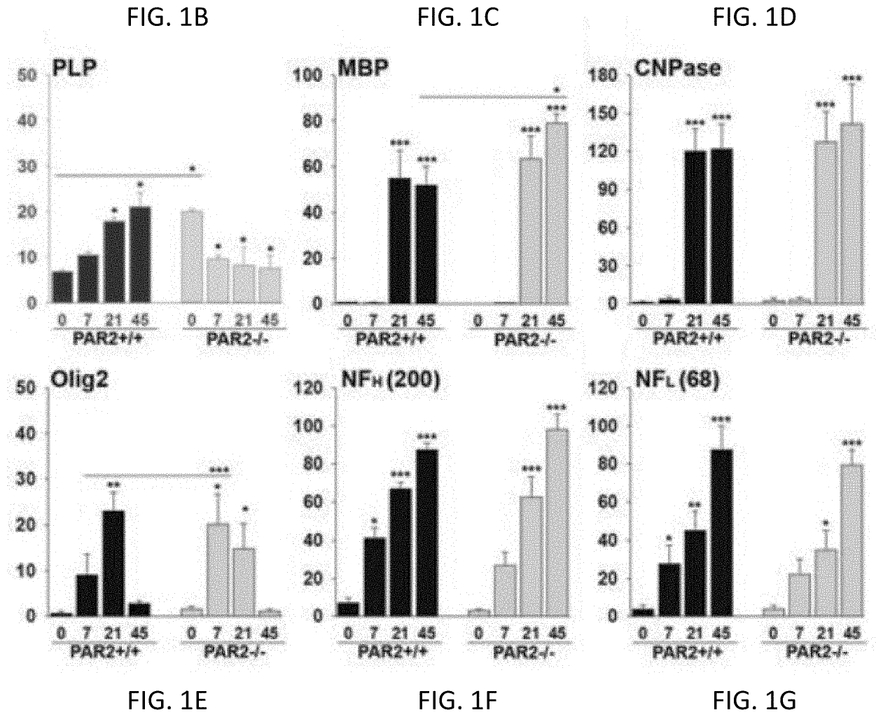

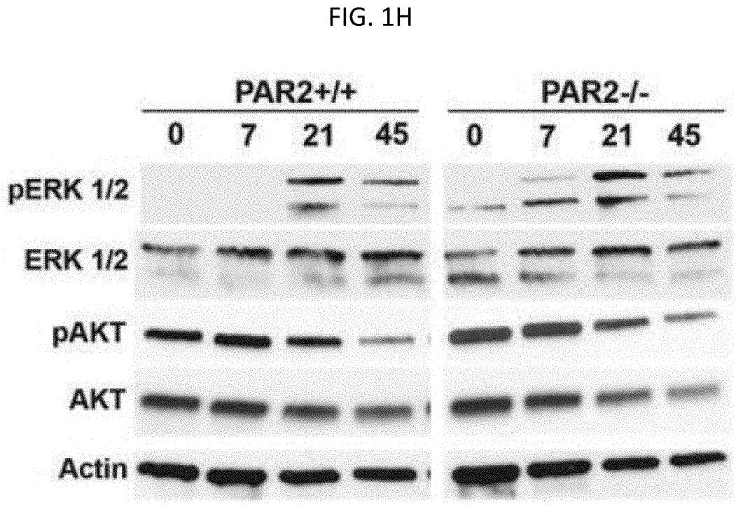

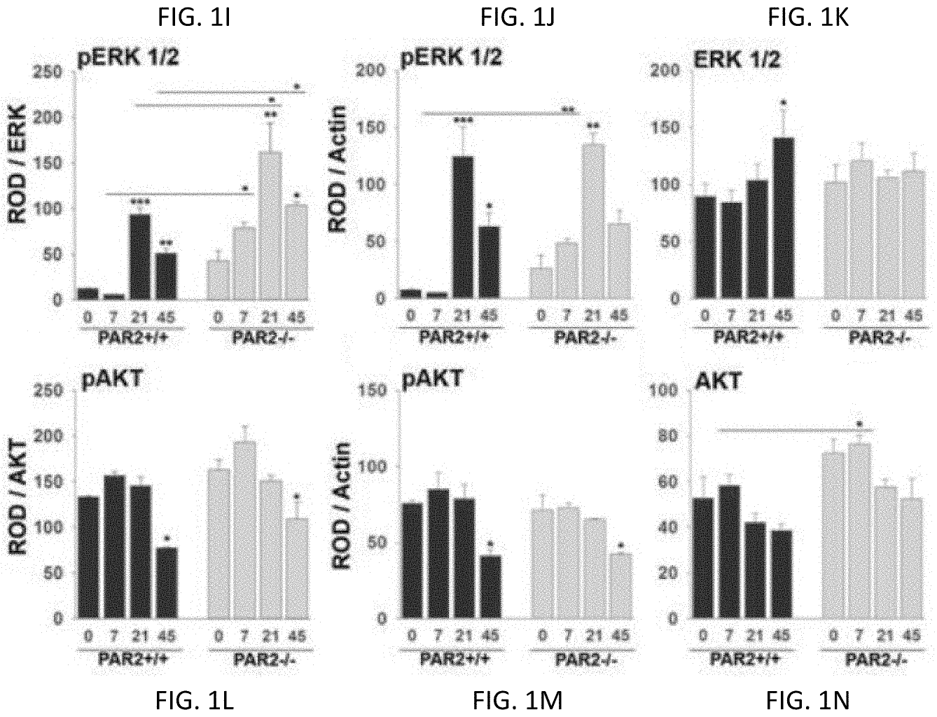

To critically evaluate the role of PAR2 loss-of-function in myelin development in vivo, the onset, magnitude, and duration of expression of myelin proteins, including the two major myelin structural proteins, proteolipid protein (PLP) and myelin basic protein (MBP), were directly compared in the spinal cord of PAR2+/+ and PAR2−/− mice at various stages of development from P0 to P45 (adulthood) (FIG. 1). Consistent with a regulatory role for PAR2 in onset of myelin protein expression, spinal cord PLP levels were 3-fold higher at P0 in PAR2−/− mice relative to PAR2+/+ mice (FIGS. 1A and 1B; P=0.04, NK). MBP protein levels were very low in both genotypes at birth, but by P45 MBP levels were 1.5-fold higher in mice lacking PAR2 relative to their wild type counterparts (FIGS. 1A and 1C; P=0.002, NK). These data highlight a key role for PAR2 in regulating the onset of myelin protein expression and the ultimate abundance of the major myelin proteins in the spinal cord developmentally. Levels of Olig2 protein were comparable between PAR2+/+ and PAR2−/− mice at birth, but by P7 were 2.2-fold higher in mice lacking PAR2 relative to PAR2+/+ mice (FIGS. 1A and 1E; P<0.001, NK). The peak of Olig2 expression was accelerated in PAR2−/− mice occurring at P7 compared to P21 in mice with an intact PAR receptor. There was a substantial loss of Olig2 protein expression after P21 in both genotypes. Substantial elevations in spinal cord 2′, 3′-cyclic-nucleotide 3′-phosphodiesterase (CNPase) also were observed between birth and P21 but no differences were observed across genotypes (FIGS. 1A and 1D). As expected, there was a progressive increase in the abundance of both the heavy and light chains of neurofilament protein (NFH or NFL) from birth through adulthood, with the changes being identical in the spinal cord of PAR2+/+ and PAR2−/− mice (FIGS. 1A, 1F, and 1G).

### Example 2

### PAR2 Loss-of-Function Increases ERK1/2 Signaling in the Developing Spinal Cord

As known regulators of myelinogenesis, the impact of PAR2 gene deletion on extracellular-signal-related kinase (ERK1/2) and AKT (protein kinase B) signaling were evaluated (Czopka et al., J Neurosci 30:12310-12322, 2010; Harrington et al., Ann Neurol 68:703-716, 2010; Guardiola-Diaz et al., Glia 60:476-486, 2012; Ishii et al., J. Neurosci 32:8855-8864, 2012; Fyffe-Maricich et al., J. Neurosci 33:18402-18408, 2013; and Ishii et al., J. Neurosci 33:175-186, 2013). Consistent with prior studies demonstrating that elevations in ERK1/2 signaling are associated with enhanced myelination, significantly higher levels of activated ERK1/2 were observed in the spinal cords of PAR2−/− mice at P7, P21, and adulthood, compared to wild type controls (FIGS. 1H, 1I, and 1J). Peak elevations in activated ERK1/2 occurred in both genotypes at P21, but at this time were 1.7-fold higher in the spinal cord of mice with PAR2 loss-of-function (FIGS. 1I and 1J; P=0.01, NK). Elevated levels of activated ERK1/2 also were detected in PAR2−/− spinal cords at P7, when levels were 12.9-fold higher than wild type (FIGS. 1I and 1J; P=0.02, Newman Keuls). There was a trend towards increased AKT signaling at P0 and P7, but only the changes in total AKT at P7 reached the level of statistical significance (FIGS. 1H, 1L, and 1M; P=0.01, NK).

### Example 3

### PAR2 Loss-of-Function Increases Oligodendrocyte Numbers in the Early Postnatal Period

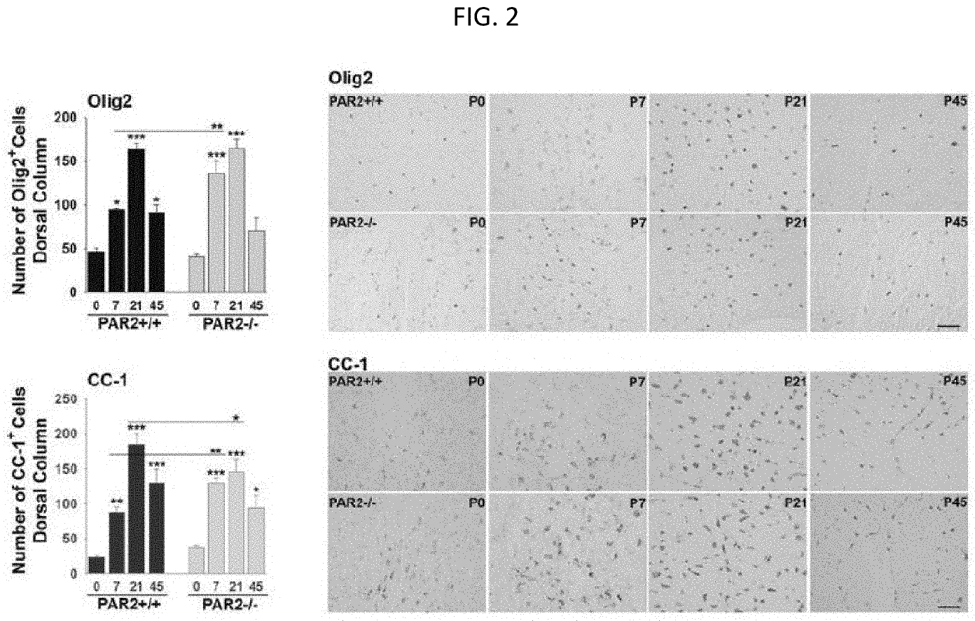

To determine whether increases in PLP and MBP protein in the spinal cord of PAR2−/− mice reflect potential increases in myelin protein expression per cell, or alternatively, more myelin producing oligodendroglia, the number of Olig2 and CC-1-immunopositive cells was quantified. The number of Olig2+ cells in the dorsal column white matter was 1.4-fold greater in PAR2−/− mice at P7 compared to wild type controls (FIG. 2, top panels; P=0.007, NK). Olig2 protein levels detected by Western blot also were higher in spinal cords of PAR2−/− mice compared to PAR2+/+ mice at P7 (2.2-fold, P<0.001, NK). The number of CC-1-immunoreactive mature oligodendrocytes also was increased 1.5-fold at P7 in the dorsal column of PAR2−/− mice (FIG. 2, bottom panels; P=0.02, NK). The number of CC-1-positive oligodendrocytes was 1.3-fold lower in PAR2−/− mice at P21 compared to PAR2+/+ mice (P<0.05, NK). The number of Olig2 and CC-1 cells in the dorsal column white matter in adult mice was identical in PAR2+/+ and PAR2−/− mice.

### Example 4

### PAR2-Loss-of-Function Enhances Expression of Myelin Proteins in Differentiated Oligodendroglia In Vitro

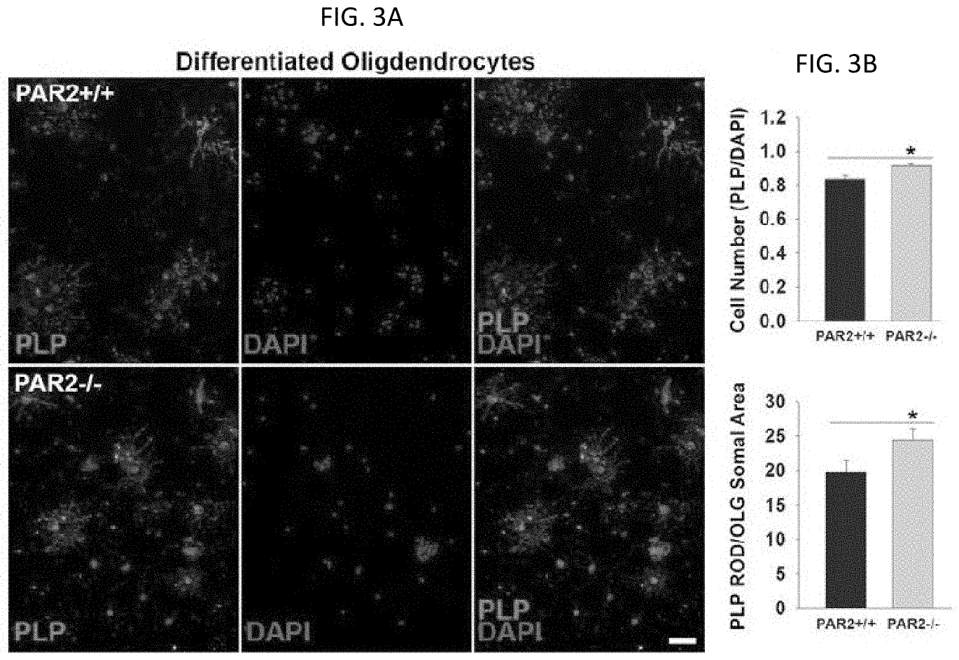

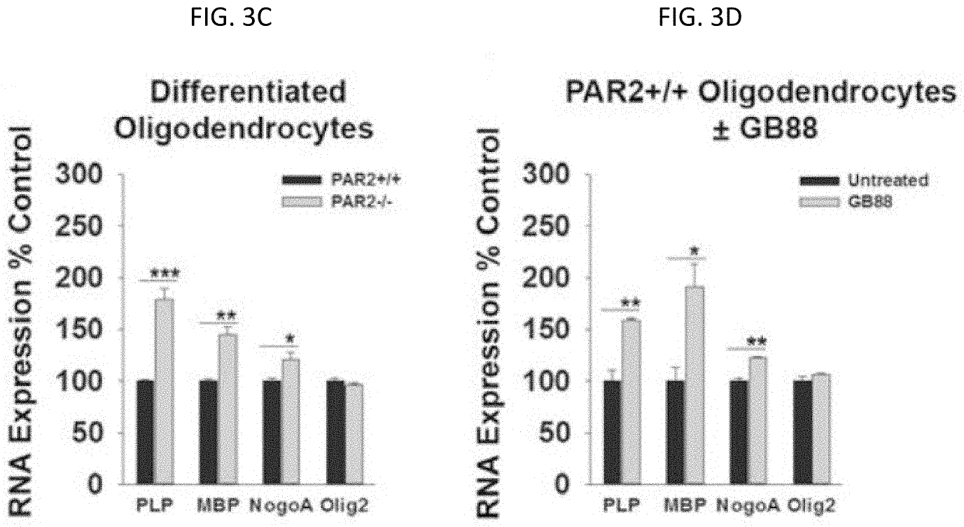

To determine whether reductions in PAR2 at the level of the oligodendrocyte directly impact myelin expression, the appearance of myelin-associated proteins was evaluated in OPCs derived from wild type or PAR2−/− mice in cell culture (FIG. 3). After a 72 hour period of differentiation, the number of oligodendrocytes immunopositive for PLP was about 10% greater in PAR2−/− compared to PAR2+/+ cultures (FIGS. 3A and 3B; P=0.01, Students t-test). In addition, oligodendrocytes lacking PAR2 expressed 1.2-fold higher levels of PLP (FIGS. 3A and 3B; P<0.05, Students t-test). Differentiated oligodendrocytes lacking PAR2 expressed higher levels of PLP (1.8-fold, P=0.002), MBP (1.5-fold, P=0.006), and NogoA RNA (FIG. 3C; 1.2-fold, P=0.02, Students t-test). Near parallel increases in myelin associated genes were observed when PAR2+/+OPCs were treated with a PAR2 small molecule inhibitor (GB88, 5 um) with 1.6-fold increases in PLP (P=0.005), 1.9-fold increases in MBP (P=0.03), and 1.2-fold increases in NogoA (P=0.002) (FIG. 3D; Student's t-test). Olig2 RNA expression levels were identical across all cultures, suggesting the increases in the expression of myelin-associated genes were a reflection of increased gene transcription rather than changes in cell abundance.

### Example 5

### PAR2 Regulates Myelin Thickness in the Adult Spinal Cord

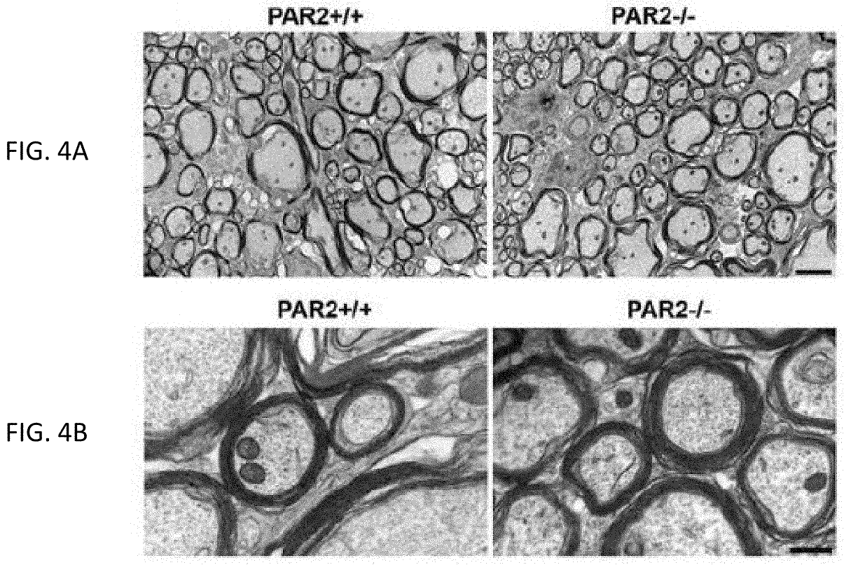

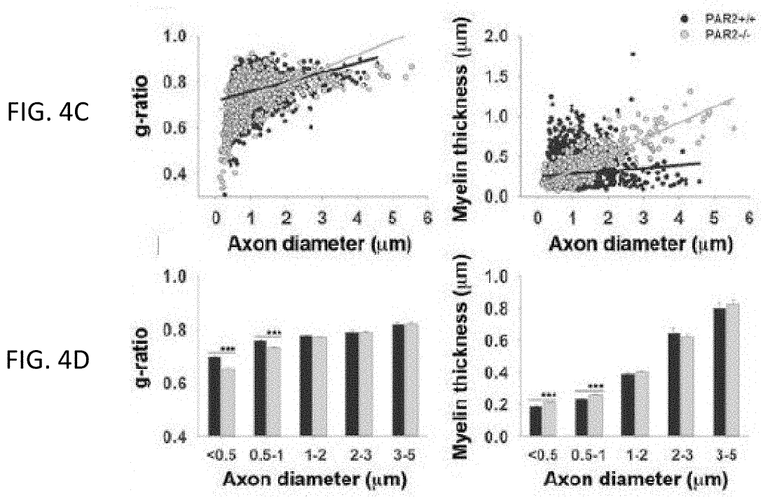

To determine whether increases in myelin protein expression in the adult spinal cord were reflected in myelin thickness, ultrastructural approaches were used to evaluate g-ratios with the dorsal column (FIGS. 4A and 4B). The myelin sheaths of mice lacking PAR2 showed significantly reduced g-ratios (0.72±0.002) compared to PAR2+/+ mice (0.75±0.002, P=0.37×10⁻⁴³, Students t-test; FIG. 4C). In addition, myelin thickness was significantly greater in mice lacking PAR2 (PAR2−/−=0.29±0.003 μm; PAR2+/+=0.28±0.003 μm; P=0.01, Students t-test; FIG. 4C). About 53 to 60 percent of all axons in the dorsal column were between 0.5 to 1 μm in diameter, and this is where the most significant increases in myelin thickness were observed (P≤1.5×10⁻⁶, Students t-test; FIG. 4D).

### Example 6

### PAR2 Loss-of-Function Enhances the Number of Oligodendrocytes in White Matter of the Adult Brain

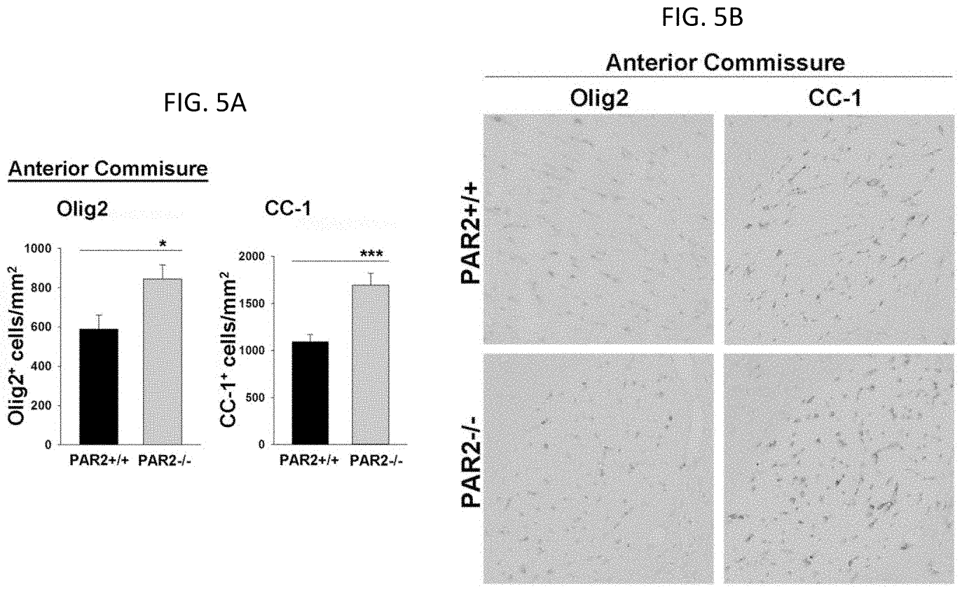

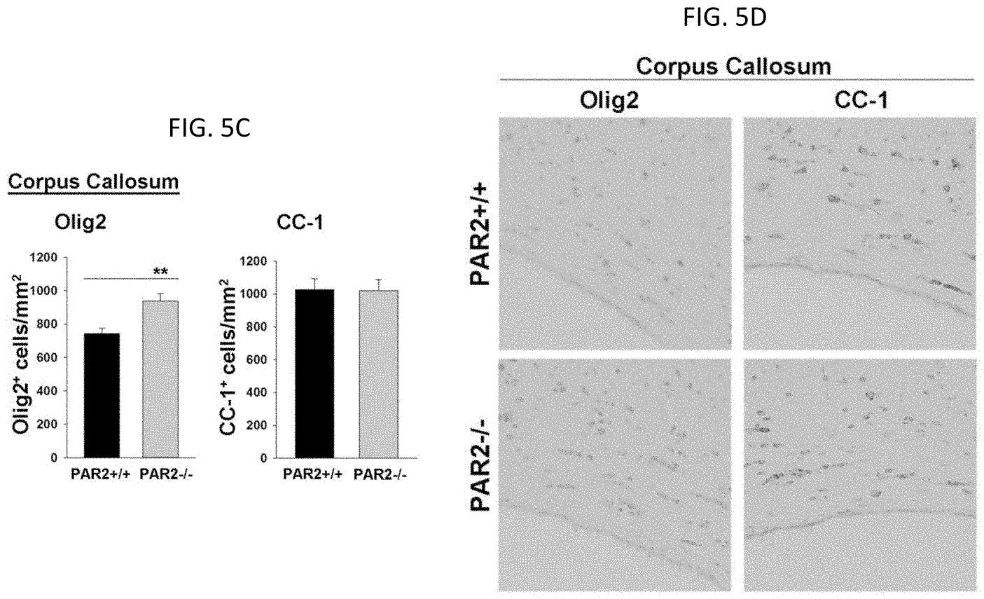

To determine whether the PAR2 loss-of-function results in enhancements in myelinating cells in the brain as it does in the adult spinal cord, counts were determined for Olig2- or CC-1-immunoreactive cells in the corpus callosum and anterior commissure of 8 week old wild type and PAR2 knockout mice. The number of Olig2+ cells was increased by 1.4-fold in the anterior commissure and 1.3-fold in the corpus callosum of PAR2−/− compared to wild type mice (P≤0.02, Students t-test; left panels of FIGS. 5A-5D). In addition, the number of CC-1+cells was increased by 1.6-fold in the anterior commissure of mice with PAR2 loss-of-function compared to wild type mice (P=0.0006, Students t-test; right panels of FIGS. 5A and 5B).

### Example 7

### PAR2 Loss-of-Function Enhances Proliferation and the Number of Neural Stem Cells in the SVZ of the Adult Brain

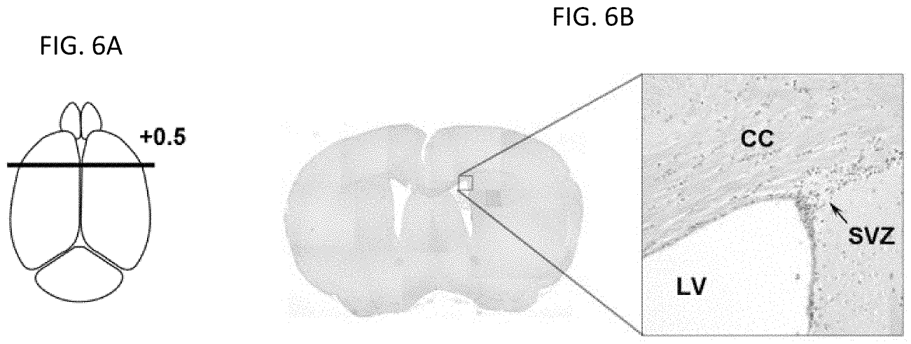

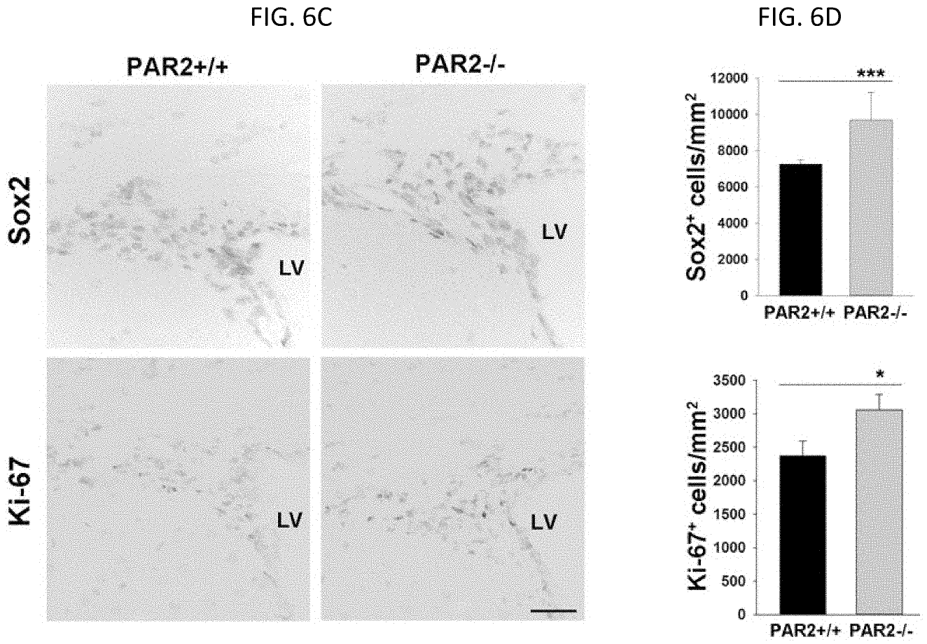

To determine whether increases in oligodendrocyte number in the corpus callosum and anterior commissure of the adult brain are reflected in changes in the SVZ, counts of Sox2+ neural stem cells in the lateral wall of the lateral ventricle +0.5 mm to Bregma were determined (FIGS. 6A and 6B). The number of Sox2+ multipotent neural stem cells per mm² was 1.3-fold greater in mice with PAR2 loss of function (P=0.0003, Students t-test; FIGS. 6C and 6D, top panels). The number of cells positive for Ki-67, a marker of proliferation, also was increased by 1.3-fold in PAR2−/− mice relative to their wild type counterparts (P=0.04, Students t-test; FIGS. 6C and 6D, bottom panels).

### Example 8

### Motor Activity in PAR2−/− Mice

To determine whether enhancements in spinal cord myelination observed in PAR2−/− mice may result in changes in motor outcomes, overall motor activity, ambulation, and rearing were evaluated during diurnal and nocturnal cycles under both fed and fasted conditions, using a comprehensive laboratory animal monitoring system.

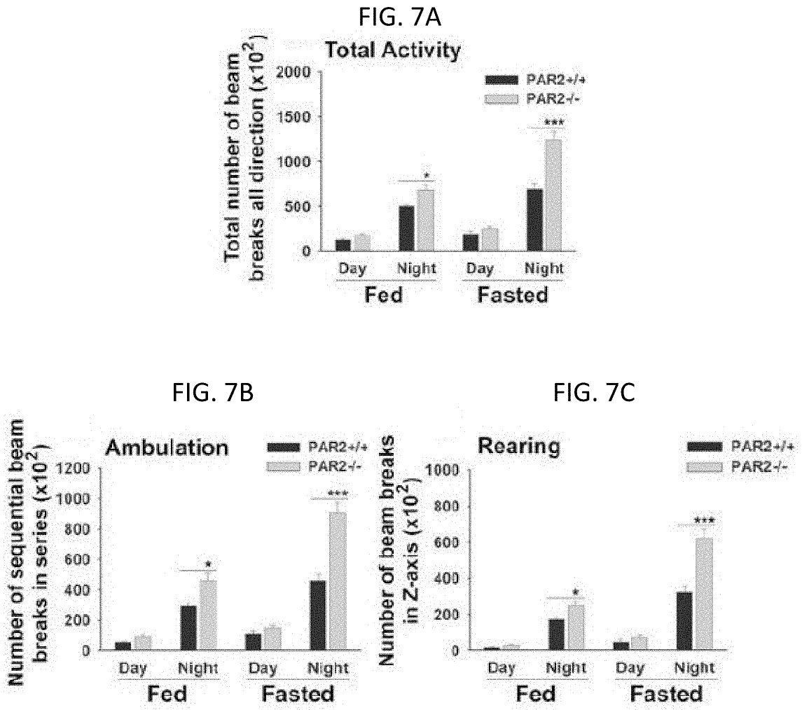

Overall activity of mice lacking PAR2 was increased by at night under fed (1.4-fold) or fasted (1.8-fold) conditions (FIG. 7A). In addition, both nocturnal ambulation and rearing responses were increased in PAR2-deficient mice under fed or fasted conditions (1.5- to 2-fold, P≤0.05, Students unpaired t-test; FIGS. 7B and 7C).

### Example 9

### PAR2 Loss-of-Function Improves Myelin Integrity After Traumatic SCI

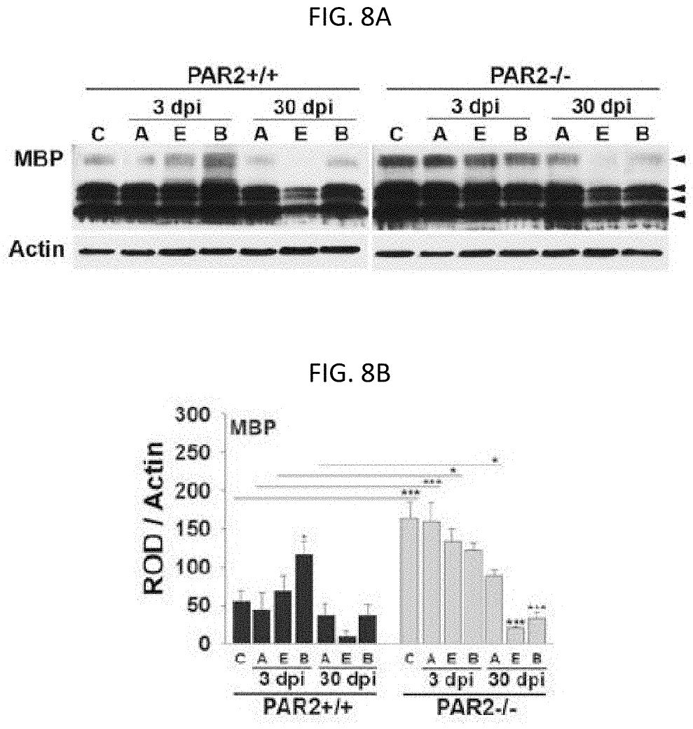

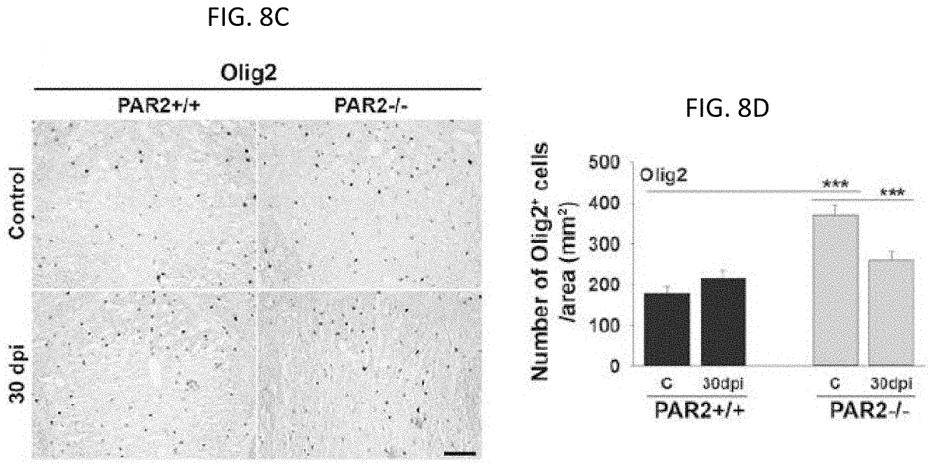

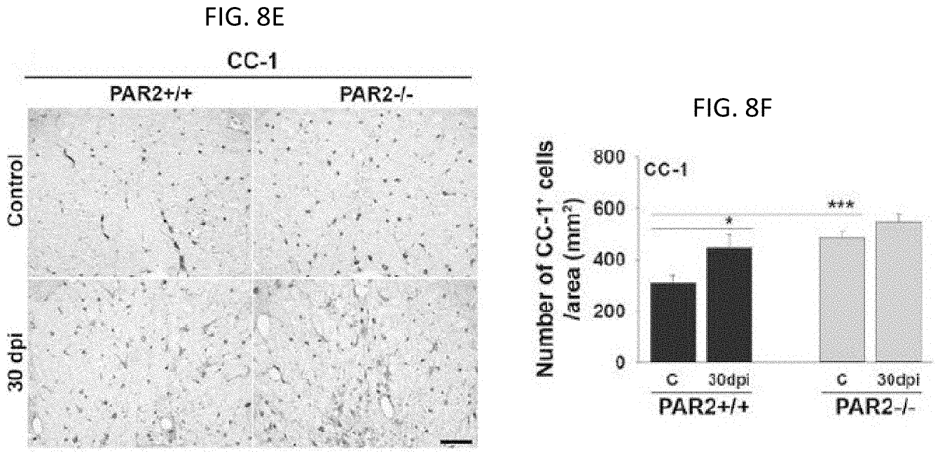

To determine whether the pro-myelination effects of PAR2 loss-of-function also occur in the context of CNS injury, the appearance of myelin markers was compared in an experimental model of contusion-compression spinal cord injury. SCI was induced in P90 mice such that all mice were P120 at the 30 dpi end point. Mirroring the 1.5-fold elevation in MBP protein observed in the intact spinal cord of P45 PAR2−/− mice relative to PAR2+/+ (FIG. 1), MBP protein levels were 3-fold higher in the uninjured spinal cord of PAR2−/− mice at P120 (P=0.001, NK; FIGS. 8A and 8B). At 3 dpi, MBP protein levels were 3.7-fold higher in spinal segments above the injury epicenter and 2-fold higher at the injury epicenter in mice with PAR2 loss-of-function compared to mice with an intact PAR2 signaling system (P<0.04, NK). At 30 dpi, MBP protein levels above the injury epicenter were 2.4-fold higher in PAR2−/− compared to wild type mice.

To determine whether differences in myelin abundance in P120 PAR2−/− mice before and after SCI were reflected in differences in the number of myelinating cells, counts of Olig2 and CC-1+ cells were made in spinal segments above the lesion epicenter. The number of Olig2+-oligodendrocytes was 2.1-fold higher in mice with PAR2 loss of function compared to wild type mice prior to injury remained 1.2-fold higher 30 dpi (P≤0.001, NK; FIGS. 8C and 8D). In parallel, the number of mature CC-1+-oligodendrocytes was 1.6-fold higher in mice with PAR2 loss-of-function prior to SCI (P=0.018, NK; FIGS. 8E and 8F).

### Example 10

### PAR2 Loss-of-Function Facilitates Myelin Regeneration

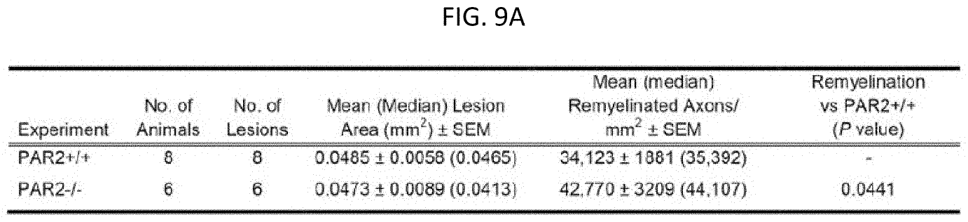

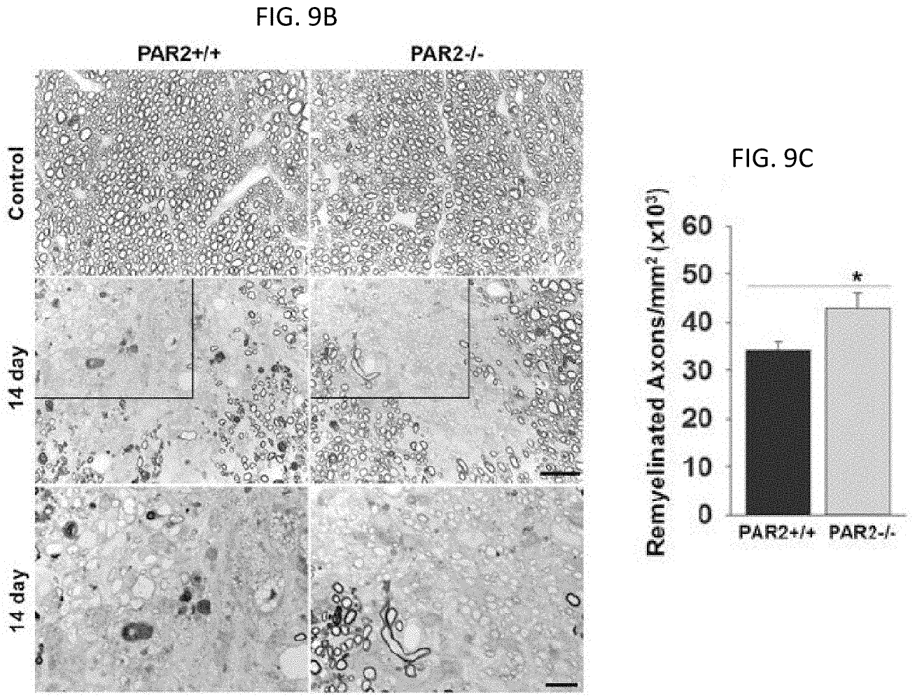

The potential impact of PAR2 loss-of-function on myelin repair in the adult spinal cord was evaluated by making counts of remyelinated axons 14 days after lysophosphatidyl choline-mediated induction of a focal demyelinating lesion in the dorsal column white matter. Similar lesion sizes in PAR2+/+ (0.049±0.005 mm²) and PAR2−/− (0.047±0.008 mm²) mice were confirmed (FIGS. 9A and 9B). The mean number of remyelinated axons in PAR2+/+ mice at 14 dpi was 34,123±1881. The mean number of remyelinated axons was increased by 25% at the same time post-lesion in mice with PAR2 loss-of-function, which had a mean of 42,770±3209 remyelinated axons (P=0.04, Students t-test; FIGS. 9A and 9C). These results suggested that inhibition of PAR2 may be a useful target to foster myelin regeneration in the adult spinal cord.

### Example 11

### Using Murine and Human Model Systems to Assess PAR2 as a Therapeutic Target for Promoting Myelin Homeostasis, Protection and Regeneration

Additional studies are conducted to evaluate the regulatory role of PAR2 in myelin development, to determine the regulatory role of PAR2 in myelin repair after toxin induced demyelination, and to determine the regulatory role of PAR2 in myelin repair in CNS inflammatory disease. To assess the role of PAR2 in regulating CNS myelin development, the timing of oligodendrocyte differentiation, axon ensheathment, myelin protein production, and myelin thickness is determined in the brain and spinal cord of mice with global PAR2 loss-of-function, with conditional deletion of PAR2 selectively in OPCs, or after administration of a PAR2 small molecule inhibitor. The impact on PAR2 loss-of-function or gain-of-function on myelin development is mechanistically dissected in vitro using murine myelinating cultures and induced pluripotent stem cell- (iPSC-) derived human three-dimensional myelinating brain aggregates.

To evaluate the activity of PAR2 as an innate suppressor of myelin regeneration, the kinetics and quality of remyelination are determined following lysolecithin injection into the corpus callosum, or into the spinal cord white matter of adult mice with global PAR2 loss-of-function, or with conditional deletion of PAR2 selectively in OPCs. Findings are extended using the Cuprizone model of CNS demyelination and myelin repair, in which myelin loss and functional deficits are more widespread, thus permitting assessment of the impact of genetic or pharmacologic PAR2 loss-of-function on recovery of neurologic function. The impact of targeting PAR2 on myelin regeneration is then mechanistically dissected in vitro using the same murine and human-derived myelinating culture platforms used to study myelination developmentally as described above.

To test the role that PAR2 plays in the failure of remyelination observed in the context of CNS inflammatory conditions such as MS, studies are conducted to critically evaluate whether conditional deletion of PAR2 specifically in OPCs facilitates remyelination in myelin oligodendrocyte glycoprotein- (MOG35-55-) induced experimental autoimmune encephalomyelitis. The findings are complemented by determination of the spatial and temporal localization of PAR2 expression in demyelinating and remyelinating MS lesions. Collectively, these studies provide disease-relevant insights for understanding the role of PAR2 in demyelinating disease, and for optimizing PAR2 targeting strategies for myelin regeneration in a clinical setting.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a mammal, wherein the method comprises administering, to a mammal identified as being in need of increased myelination, increased remyelination, increased myelin protection, or increased myelin preservation, an agent that reduces the activity of protease activated receptor 2 (PAR2), or a composition comprising an agent that reduces the activity of PAR2, wherein the agent or the composition is administered in an amount effective to increase myelination, increase remyelination, or reduce demyelination in the mammal.

2. The method of claim 1, wherein the agent is a small molecule inhibitor of PAR2, an antibody against PAR2, an inhibitory RNA, or an antisense nucleic acid molecule.

3. The method of claim 1, wherein the mammal is a human.

4. The method of claim 3, wherein the human is a preterm infant.

5. The method of claim 3, wherein the human is an adult.

6. The method of claim 1, wherein the mammal is identified as having a central nervous system (CNS) demyelinating condition.

7. The method of claim 6, wherein the CNS demyelinating condition is a CNS injury, multiple sclerosis (MS), amyotrophic lateral sclerosis (ALS), Alzheimer's disease (AD), a spinal cord injury, a neuropsychiatric disorder, or stroke.

8. A method for promoting differentiation of an oligodendrocyte precursor cell (OPC), comprising contacting the OPC with an agent that reduces the activity of PAR2, or with a composition containing an agent that reduces the activity of PAR2.

9. The method of claim 8, wherein the agent is a small molecule inhibitor of PAR2, an antibody against PAR2, an inhibitory RNA, or an antisense nucleic acid molecule.

10. The method of claim 8, wherein the OPC is in vivo.

11. The method of claim 10, wherein the OPC is in a mammal.

12. The method of claim 11, wherein the mammal is identified as having a CNS demyelinating condition.

13. The method of claim 12, wherein the CNS demyelinating condition is a CNS injury, MS, ALS, AD, a spinal cord injury, a neuropsychiatric disorder, or stroke.

14. A method for treating a mammal, wherein the method comprises administering, to a mammal identified as being in need of increased numbers of oligodendrocytes, an agent that reduces the activity of PAR2, or a composition containing an agent that reduces the activity of PAR2, wherein the agent or the composition is administered in an amount effective to increase the number of oligodendrocytes in the mammal.

15. The method of claim 14, wherein the agent is a small molecule inhibitor of PAR2, an antibody against PAR2, an inhibitory RNA, or an antisense nucleic acid molecule.

16. The method of claim 14, wherein the mammal is a human.

17. The method of claim 14, wherein the mammal is identified as having a CNS demyelinating condition.

18. The method of claim 17, wherein the CNS demyelinating condition is a CNS injury, MS, ALS, AD, a spinal cord injury, a neuropsychiatric disorder, or stroke.

19. A method for treating a CNS demyelinating condition in a mammal in need thereof, wherein the method comprises administering, to said mammal, a plurality of modified stem cells that have reduced PAR2 expression as compared to corresponding wild type stem cells, and wherein the composition comprises an amount of modified stem cells that is effective to reduce or prevent demyelination, or to enhance myelination or remyelination, when administered to said mammal.

20. The method of claim 19, wherein the modified stem cells are neural stem cells having a mutation in the PAR2 gene.

21. The composition of claim 19, wherein the CNS demyelinating condition is a CNS injury, MS, ALS, AD, a spinal cord injury, a neuropsychiatric disorder, or stroke.

22. The method of claim 19, wherein the mammal is a human.
